(19) United States
(12) Patent Application Publication
(10) Pub. No.: US 2010/0068701 A1
(43) Pub. Date: Mar. 18, 2010

(54) CHROMOSOME LABELING METHOD

(76) Inventors: N. Alice Yamada, San Jose, CA (US); Amir Ben-Dor, Kfar Kava (IL)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 12/210,094

(22) Filed: Sep. 12, 2008

(51) Int. Cl. C12Q 1/68

(52) U.S. Cl. 435/6

(57) ABSTRACT

A method of sample analysis is provided. In certain embodiments, the method may involve a) contacting a genomic sample comprising a test chromosome with a plurality of sets of labeled oligonucleotide probes under in situ hybridization conditions to produce a contacted sample having an oligonucleotide binding pattern; b) imaging the contacted sample to provide an image showing the oligonucleotide binding pattern; and c) analyzing the oligonucleotide binding pattern to identify a chromosomal rearrangement in the test chromosome relative to a reference chromosome.

### BACKGROUND

Chromosomal rearrangements and aberrations are a type of genomic variation, which have been long been associated with genetic diseases. Numerical abnormalities, also known as aneuploidy, may occur as a result of nondisjunction during meiosis in the formation of a gamete. Trisomies, in which three copies of a chromosome are present instead of the usual two, are a common numerical abnormality seen in Edwards, Patau and Down syndromes. Structural abnormalities often arise from errors in homologous recombination. Both types of abnormalities can occur in gametes and therefore will be present in all cells of an affected person's body, or they can occur during mitosis and give rise to a genetic mosaic individual who has some normal and some abnormal cells.

Genomic instability also leads to complex patterns of chromosomal rearrangements in certain cells, such as cancer cells, for example. Standard cytogenetic assays such as Giemsa (G) banding have identified numerous cancer-specific translocations and chromosomal abnormalities in cancer cells such as the Philadelphia (t9,22) chromosome. Down syndrome (a trisomy), Jacobsen syndrome (a deletion) and Burkitt's lymphoma (a translocation) have traditionally been studied via karyotype analysis.

Improvements in cytogenetic banding and visualization such as M banding and spectral karyotyping (SKY) have enabled detailed analyses on a chromosome by chromosome basis of inversions and translocations, as well as the identification of unbalanced gain or loss of chromosomal material in cancers of interest. Fluorescence in situ hybridization (FISH) further allows for the detection of the presence or absence of specific DNA sequences on chromosomes by using fluorescent probes that bind to only those parts of the chromosome with which they show a high degree of complementarity.

All of these methods, however, have limited resolution since probes are generated from large pieces of DNA (flow-sorted chromosomes or bacterial artificial chromosomes for SKY and FISH, respectively). Because probes are generated over very large regions of the genome, microtranslocations and microinversions cannot be resolved by current methods. The large templates from which probes are generated also presents another disadvantage, in that both SKY and FISH probes contain repetitive DNA elements that are inherent in the large template DNA fragments. Thus, there has been an increasing need to understand more subtle chromosomal defects with substantially improved resolution, and without a priori knowledge of their location. A large unmet need exists to develop technical methods that detect novel, specific chromosomal abnormalities.

Certain aspects of this disclosure describe methods for detecting chromosomal rearrangements, such as inversions and translocations, and kits for practicing the same.

### SUMMARY

A method of sample analysis is provided. In certain embodiments, the method may involve: a) contacting a genomic sample comprising a test chromosome with a plurality of sets of labeled oligonucleotide probes under in situ hybridization conditions to produce a contacted sample having an oligonucleotide binding pattern; b) imaging the contacted sample to provide an image showing the oligonucleotide binding pattern; and c) analyzing the oligonucleotide binding pattern to identify a chromosomal rearrangement in the test chromosome relative to a reference chromosome. In general terms: i. each set of labeled oligonucleotide probes comprises at least 100 different labeled oligonucleotide probes, ii. the labeled oligonucleotide probes of each set bind to a plurality of distinct non-contiguous regions of a reference chromosome that is used for comparison purposes, iii. the plurality of sets of labeled oligonucleotide probes bind to the reference chromosome in a predetermined binding pattern, and iv. each set of labeled oligonucleotide probes is labeled so as to produce an optically detectable signature that is distinguishable from all other sets. Kits and compositions for practicing the method are also provided.

### BRIEF DESCRIPTION OF THE FIGURES

FIG. 1 schematically illustrates certain features of an embodiment of a method for sample analysis described herein.

### DEFINITIONS

The term “sample” as used herein relates to a material or mixture of materials, typically, although not necessarily, in liquid form, containing one or more analytes of interest.

The term “genomic sample” as used herein relates to a material or mixture of materials, containing genetic material from an organism. The term “genomic DNA” as used herein refers to deoxyribonucleic acids that are obtained from an organism. The terms “genomic sample” and “genomic DNA” encompass genetic material that may have undergone amplification, purification, or fragmentation. The term “test genome,” as used herein refers to genomic DNA that is of interest in a study.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively).

The term “oligonucleotide” as used herein denotes a single stranded multimer of nucleotide of from about 2 to 200 or more, up to about 500 nucleotides or more. Oligonucleotides may be synthetic or may be made enzymatically, and, in some embodiments, are less than 10 to 50 nucleotides in length. Oligonucleotides may contain ribonucleotide monomers (i.e., may be oligoribonucleotides) or deoxyribonucleotide monomers. Oligonucleotides may be 10 to 20, 11 to 30, 31 to 40, 41 to 50, 51-60, 61 to 70, 71 to 80, 80 to 100, 100 to 150 or 150 to 200 nucleotides in length, for example.

The term “sequence-specific oligonucleotide” as used herein refers to an oligonucleotide that only binds to a single site in a haploid genome. In certain embodiments, a “sequence-specific” oligonucleotide may hybridize to a complementary nucleotide sequence that is unique in a sample under study.

The term “complementary” as used herein refers to a nucleotide sequence that base-pairs by non-covalent bonds to a target nucleic acid of interest. In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is fully complementary to a target of interest such that every nucleotide in the sequence is complementary to every nucleotide in the target nucleic acid in the corresponding positions. In certain cases, a nucleotide sequence may be partially complementary to a target, in which not all nucleotide is complementary to every nucleotide in the target nucleic acid in all the corresponding positions.

The term “probe,” as used herein, refers to a nucleic acid that is complementary to a nucleotide sequence of interest. In certain cases, detection of a target analyte requires hybridization of a probe to a target. In certain embodiments, a probe may be immobilized on a surface of a substrate, where the substrate can have a variety of configurations, e.g., a sheet, bead, or other structure. In certain embodiments, a probe may be present on a surface of a planar support, e.g., in the form of an array.

An “array,” includes any two-dimensional or substantially two-dimensional (as well as a three-dimensional) arrangement of addressable regions, e.g., addressable regions, e.g., spatially addressable regions or optically addressable regions, bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a surface of the substrate. Depending upon the use, any or all of the arrays may be the same or different from one another and each may contain multiple spots or features. An array may contain one or more, including more than two, more than ten, more than one hundred, more than one thousand, more ten thousand features, more than one hundred thousand features, or even more than million features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 mm and less than 1.5 mm, such as more than about 0.8 mm and less than about 1.2 mm.

Arrays can be fabricated using drop deposition from pulse-jets of either precursor units (such as nucleotide or amino acid monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

Arrays may also be made by distributing pre-synthesized nucleic acids linked to beads, also termed microspheres, onto a solid support. In certain embodiments, unique optical signatures are incorporated into the beads, e.g. fluorescent dyes, which could be used to identify the chemical functionality on any particular bead. Since the beads are first coded with an optical signature, the array may be decoded later, such that correlation of the location of an individual site on the array with the probe at that particular site may be made after the array has been made. Such methods are described in detail in, for example, U.S. Pat. Nos. 6,355,431, 7,033,754, and 7,060,431.

An array is “addressable” when it has multiple regions of different moieties (e.g., different oligonucleotide sequences) such that a region (i.e., a “feature” or “spot” of the array) at a particular predetermined location (i.e., an “address”) on the array contains a particular sequence. Array features are typically, but need not be, separated by intervening spaces. An array is also “addressable” if the features of the array each have an optically detectable signature that identifies the moiety present at that feature.

The terms “determining”, “measuring”, “evaluating”, “assessing”, “analyzing”, and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

The term “chromosomal rearrangement,” as used herein, refers to an event where one or more parts of a chromosome are rearranged within a single chromosome or between chromosomes. In certain cases, a chromosomal rearrangement may reflect an abnormality in chromosome structure. A chromosomal rearrangement may be an inversion, a deletion, an insertion or a translocation, for example.

The term “contacting” means to bring or put together. As such, a first item is contacted with a second item when the two items are brought or put together, e.g., by touching them to each other or combining them in the same solution. Thus, a “contacted sample” is a test chromosome onto which oligonucleotide probes have been hybridized.

The term “hybridization” refers to the specific binding of a nucleic acid to a complementary nucleic acid via Watson-Crick base pairing. Accordingly, the term “in situ hybridization” refers to specific binding of a nucleic acid to a metaphase or interphase chromosome.

The terms “hybridizing” and “binding”, with respect to nucleic acids, are used interchangeably.

The terms “plurality”, “set” or “population” are used interchangeably to mean at least 2, at least 10, at least 100, at least 500, at least 1000, at least 10,000, at least 100,000, at least 1000,000, at least 10,000,000 or more.

The term “chromosomal region” as used herein denotes a contiguous length of nucleotides in a genome of an organism. A chromosomal region may be in the range of 10 kb in length to an entire chromosome, e.g., 100 kb to 10 MB for example.

A “test chromosome” is an intact metaphase or interphase chromosome isolated from a mammalian cell, where an intact chromosome has the same overall morphology as the same chromosome present in the mammalian cell, e.g., contains a centromere, a long arm containing a telomere and a short arm containing a telomere. A test chromosome may contain an inversion, translocation, deletion insertion, or other rearrangement relative to a reference chromosome. A test chromosome is the chromosome under study.

A “reference chromosome” is an intact metaphase chromosome to which a test chromosome may be compared to identify a rearrangement. A reference chromosome may be arbitrarily chosen. A reference chromosome may have a known sequence. A reference chromosome may itself contain a chromosomal rearrangement.

The term “reference chromosomal region,” as used herein refers to a chromosomal region to which a test chromosomal is compared. In certain cases, a reference chromosomal region may be of known nucleotide sequence, e.g., a chromosomal region whose sequence is deposited at NCBI's Genbank database or other database, for example.

The term “in situ hybridization conditions” as used herein refers to conditions that allow hybridization of a nucleic acid to a complementary nucleic acid in an intact chromosome. Suitable in situ hybridization conditions may include both hybridization conditions and optional wash conditions, which include temperature, concentration of denaturing reagents, salts, incubation time, etc. Such conditions are known in the art.

“Distinct non-contiguous regions” refers to regions or intervals on a chromosome that are not contiguous.

A “binding pattern” refers to the pattern of binding of a set of labeled probes to an intact chromosome.

### DESCRIPTION OF EXEMPLARY EMBODIMENTS

A method of sample analysis is provided. In certain embodiments, the method may involve a) contacting a genomic sample comprising a test chromosome with a plurality of sets of labeled oligonucleotide probes under in situ hybridization conditions to produce a contacted sample having an oligonucleotide binding pattern, where i. each set of labeled oligonucleotide probes comprises at least 100 different labeled oligonucleotide probes, ii. the labeled oligonucleotide probes of each set bind to a plurality of distinct non-contiguous regions of a reference chromosome that is used for comparison purposes, iii. the plurality of sets of labeled oligonucleotide probes bind to the reference chromosome in a predetermined binding pattern, and iv. each set of labeled oligonucleotide probes is labeled so as to produce an optically detectable signature that is distinguishable from all other sets; b) imaging the contacted sample to provide an image showing the oligonucleotide binding pattern; and c) analyzing the oligonucleotide binding pattern to identify a chromosomal rearrangement in the test chromosome relative to a reference chromosome.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Method for Sample Analysis

As noted above, the instant method uses a plurality of sets of labeled oligonucleotides, where: i. each set of labeled oligonucleotide probes comprises at least 100 different labeled oligonucleotide probes, ii. the labeled oligonucleotide probes of each set bind to a plurality of distinct non-contiguous regions of a reference chromosome, iii. the plurality of sets of labeled oligonucleotide probes bind to the reference chromosome in a predetermined binding pattern, and iv. each set of labeled oligonucleotide probes is labeled so as to produce an optically detectable signature that is distinguishable from all other sets.

The number of sets of labeled oligonucleotides, the number of labeled oligonucleotides within each set, the sequences of the labeled oligonucleotides and the label of each of the sets of labeled oligonucleotides is flexible and may be determined by the complexity of the desired chromosome banding. However, in certain embodiments, there may be at least 2, at least 3, at least 4, at least 5, at least 10, at least 50, or at least 100 or more sets of labeled oligonucleotides used the method. Within each set of labeled oligonucleotides and in certain embodiments, there may be at least 10 different labeled oligonucleotides, e.g., at least 50, at least 100, at least 500, at least 1,000, at least 5,000, at least 10,000 or more, up to 50,000 or 100,000 or more different labeled oligonucleotides. Depending on the desired result, the oligonucleotides may be from about 15 nucleotides to about 200 nucleotides in length, or greater, e.g., 20 nucleotides to 80 nucleotides or 25 nucleotides to 50 nucleotides in length, as desired.

In particular embodiments, the different sets of labeled oligonucleotides are distinguishably labeled so that they can be distinguished from one other. In one embodiment, the oligonucleotides of a set may each be labeled with a single label. For example, a first set of oligonucleotides may be labeled with a first label, a second set of oligonucleotides may be labeled with a second label that is distinguishable from the first label, and a third set may be labeled with a third label that is distinguishable from the first and second labels. In another embodiment, each set of oligonucleotides may be labeled with two or more labels, where either the combination of the labels (i.e., the identities of the labels associated with the oligonucleotides) or the ratio of the magnitudes of the signals from the labels identifies the labeled oligonucleotides. In an exemplary embodiment, a first set of oligonucleotides may be labeled with Cy3 only, and another set of oligonucleotides may be labeled with Cy3 and Cy5, where the single signal obtained from the Cy3 only-labeled oligonucleotides is distinguishable from the composite signal obtained from the Cy3/Cy5-labeled oligonucleotides. Likewise, a set of oligonucleotides labeled with 80% Cy3 and 20% Cy5 is distinguishable from a set of oligonucleotides labeled with 20% Cy3 and 80% Cy5 by the ratio of the magnitudes of the signals produced by the labels.

In certain embodiments, each species of labeled oligonucleotide (i.e., the labeled oligonucleotides that have the same nucleotide sequence) of a set of labeled oligonucleotides specifically bind to a unique sequence (i.e., to only one position) in a reference genome, and the different species of labeled oligonucleotide may bind to different unique sequences in the reference genome. The labeled oligonucleotides of a single set of labeled oligonucleotides may all bind to one region of a genome, e.g., a region of a chromosome (e.g., a chromosomal region in the range of a 50 kb to 500 Mb in size up to a chromosome arm or an entire chromosome), or many discontinuous regions of a genome, which regions may be all on one chromosome or spread throughout many chromosomes (e.g., at least 2, at least 3, at least 4, at least 5, at least 6 or more, up to the entire complement of chromosomes of a cell). In certain embodiments, within each chromosomal region, the binding sites for the labeled oligonucleotides may be tiled such that there is an overlap between adjacent oligonucleotides (such that there is, for example, a 10% to 90% overlap between the oligonucleotides, when bound) or they may be tiled end-to-end such that the 5′ end of one oligonucleotide is next to the 3′ end of the adjacent oligonucleotide, when bound. In another embodiment, the binding sites for the oligonucleotides may be separated and interspersed within the chromosomal region.

In the plurality of sets of oligonucleotides used in the assay, any two sets of labeled oligonucleotides may bind to: a) non-overlapping, distinct regions of a genome (in which case the distinct regions will be associated with either but not both of the different labels used to label the different sets of oligonucleotides); b) the same regions of a genome (to provide a composite signal containing signals from the different labels used to label the different sets of oligonucleotides) or c) some overlapping regions and some same regions (so that some chromosomal regions will have a composite signal and others will have a single signal, for example). In certain embodiments, no two oligos used in the method bind the same binding site.

In cases in which the labeled oligonucleotides of two sets of oligonucleotides bind to the same chromosomal region, the oligonucleotides may be labeled such that the combination of the labels (i.e., the identities of the labels associated with the chromosomal region when the oligonucleotides are bound) or the ratio of the magnitudes of the signals from the labels identifies the chromosomal region. In an exemplary embodiment, if a chromosomal region is bound by some oligonucleotides that are labeled with Cy5 only and also other oligonucleotides that are labeled with Cy3 only, the composite Cy3/Cy5 signal identifies the binding site for those oligos and therefore identifies that region as different from the region bound by the only the Cy3-labeled oligonucleotides. Likewise, if 80% of the oligonucleotides that bind to a particular chromosomal region are labeled with Cy3 and 20% of the oligonucleotides that bind to that chromosomal region are labeled with Cy5, that chromosomal region can be identified by the ratio of the labels (and distinguished from, for example, a chromosomal region that is bound by other ratios of oligonucleotides, e.g., 20% labeled with Cy3 and 80% labeled with Cy5).

The chromosomal regions to which the labeled oligonucleotides bind may be of a defined size (e.g., in the range of 50 kb to 100 kb, 100 kb to 500 kb, 500 kb to 1 Mb, 1 Mb to 5 Mb, 5 Mb to 10 Mb or 10 Mb to 50 Mb, etc) and, in a single assay, the labeled oligonucleotides may hybridize to and label several different-sized chromosomal regions. As such, in addition to the chromosomal regions being labeled with different labels, different combinations of labels and different ratios of labels, the chromosomal regions may also be of different, defined, sizes. At least 10, at least 50, at least 100, at least 500, or at least 10,000 up to 50,000 or 100,000 or more distinct chromosomal regions may be targeted by the labeled oligonucleotides, as desired.

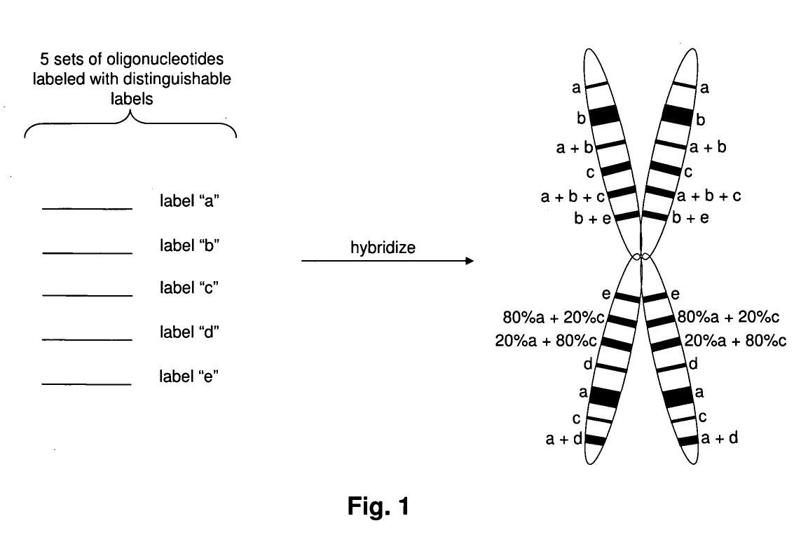

The general principles of certain aspects of the instant method are illustrated in FIG. 1. With reference to FIG. 1, a total of five sets of oligonucleotides are designed to specifically hybridize to various chromosomal regions and each set is labeled with a different distinguishable label “a”, “b”, “c”, “d” and “e”. The labeled oligonucleotides are hybridized to an intact chromosome under in situ hybridization conditions and the labeled chromosome imaged. As shown in FIG. 1, certain non-contiguous chromosomal regions may hybridize with only oligonucleotides from a single set of labeled oligonucleotides (e.g., the a, b, c, d and e regions), whereas other non-contiguous chromosomal regions may hybridize to oligonucleotides from more than one set of labeled oligonucleotides (e.g., the a+b, a+b+c, a+d, 80% a+20% c and 20% c+80% c regions). Some of those regions, although hybridized to similarly labeled oligonucleotides (i.e., the 80% a+20% c and 20% a+80% c regions) can be distinguished in that the ratios of the labels are different in the different bands. Adding further labels, further sets of oligonucleotides and further chromosomes to the method increases the amount of data that can be obtained from an assay. Certain aspects of this method offer almost limitless flexibility in terms of the banding pattern that can be obtained, e.g., in terms of the number of discrete bands, different “colors” of bands, different band sizes, band density and resolution that can be obtained from a single assay.

In one straightforward embodiment, the sets of oligonucleotides are designed such that the different chromosomes in a sample will be labeled with different colors (i.e., where a “color” is determined by the label(s) associated with the different chromosomes) that distinguish one chromosome from another and thus allow translocations to be readily identified.

In certain embodiments, the oligonucleotides may be designed to bind to pre-determined regions of a reference chromosome, e.g., a chromosome of known nucleotide sequence, and tested to provide a pre-determined binding pattern to which the binding pattern for a test chromosome may be compared in order to identify a chromosomal rearrangement, e.g., an inversion, translocation, duplication, deletion or other complex rearrangement relative to the reference chromosome.

Since the genome sequences of many organisms, including many bacteria, fungi, plants and animals, e.g., mammals such as human, primates, and rodents such as mouse and rat, are known and some are publicly available (e.g., in NCBI's Genbank database), the design of the above-described oligonucleotides is within the skill of one of skilled in the art. In particular embodiments, the variable domains of the oligonucleotides may be designed using methods set forth in US20040101846, U.S. Pat. No. 6,251,588, US20060115822, US20070100563, US20080027655, US20050282174, patent application Ser. No. 11/729,505, filed March 2007 and patent application Ser. No. 11/888,059, filed Jul. 30, 2007 and references cited therein, for example.

In certain embodiments, the oligonucleotides may be synthesized in an array using in situ synthesis methods in which nucleotide monomers are sequentially added to a growing nucleotide chain that is attached to a solid support in the form of an array. Such in situ fabrication methods include those described in U.S. Pat. Nos. 5,449,754 and 6,180,351 as well as published PCT application no. WO 98/41531, the references cited therein, and in a variety of other publications. In one embodiment, the oligonucleotide composition may be made by fabricating an array of the oligonucleotides using in situ synthesis methods, and cleaving oligonucleotides from the array. In particular embodiments, each set of oligonucleotides is made on a different array (i.e., so that there is the same number of arrays as sets of oligonucleotides), cleaved from the array and then labeled, although other methods are envisioned.

The oligonucleotides may be labeled by any of a number of means well known to those of skill in the art. For example, the label may be simultaneously incorporated during the amplification step. Means of attaching labels to nucleic acids are well known to those of skill in the art and include, for example, random priming, end-labeling, by kinasing of the nucleic acid and subsequent attachment of a nucleic acid linker joining the oligonucleotides to a label. Standard methods may be used for labeling the oligonucleotide, for example, as set out in Ausubel, et al, (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.). In one embodiment, the label may be added during synthesis of the oligonucleotide.

In general terms, once labeled, the labeled oligonucleotides are hybridized to a sample containing intact chromosomes, and the binding pattern analyzed. For example, an interphase or metaphase chromosome preparation may be produced. The chromosomes are attached to a substrate, e.g., glass, The probe is then applied to the chromosome DNA and incubated under hybridization conditions. Wash steps remove all unhybridized or partially-hybridized labeled oligonucleotides, and the results are visualized and quantified using a microscope that is capable of exciting the dye and recording images.

Such methods are generally known in the art and may be readily adapted for use herein. For example, the following references discuss chromosome hybridization: Ried et al., Chromosome painting: a useful art Human Molecular Genetics, Vol 7, 1619-1626; Speicher et al: Karyotyping human chromosomes by combinatorial multi-fluor FISH, Nature Genetics, 12, 368-376, 1996; Schröck et al: Multicolor Spectral Karyotyping of Human Chromosomes. Science, 494-497, 1996; Griffin et al Molecular cytogenetic characterization of pancreas cancer cell lines reveals high complexity chromosomal alterations. Cytogenet Genome Res. 2007; 118(2-4):148-56; Peschka et al, Analysis of a de novo complex chromosome rearrangement involving chromosomes 4, 11, 12 and 13 and eight breakpoints by conventional cytogenetic, fluorescence in situ hybridization and spectral karyotyping. Prenat Diagn. 1999 December; 19(12):1143-9; Hilgenfeld et al, Analysis of B-cell neoplasias by spectral karyotyping (SKY). Curr Top Microbiol Immunol. 1999; 246:169-74. Ried et al, Genomic changes defining the genesis, progression, and malignancy potential in solid human tumors: a phenotype/genotype correlation. Genes Chromosomes Cancer. 1999 July; 25(3):195-204; and Agarwal et al, Comparative genomic hybridization analysis of human parathyroid tumors. Cancer Genet Cytogenet. 1998 Oct. 1; 106(1):30-6.

As noted above, in certain embodiments, each set of oligonucleotides may be labeled with a fluorophore that is different from the fluorophore used to label other sets of oligonucleotides. This allows for fine-tune control over which probe is labeled with which fluorophore.

There is no requirement for blocks of genomic regions to be painted in one color to be in one contiguous region. A single chromosome can be labeled as desired, in different colors, (e.g., up to 10 different colors), and at any position (e.g., up to 100 different positions). Patterns may include, but are not limited to, longitudinal or latitudinal stripes; solid transverse bands and lighter-colored interbands, “dots”, overlapping segments, and repeats.

The lack of requirement for contiguous regions allows for the creation of new colors from standard fluorophores. As depicted in FIG. 1, genomic regions may be labeled with different fluorophores to provide different colors or different hues of similar colors. Thus, the labeled oligonucleotides may be hybridized to target nucleic acids within fixed chromosomes to provide not only complex patterns which are not readily achievable by conventional methods, but also new colors generated by different combinations of fluorophores.

Accordingly, some of the features and advantages of certain embodiments of the subject methods include: 1) avoidance of non-specific amplification of starting materials, which leads to random amplification bias; 2) consistent creation of probes of a designated length (fragments generated in current PCR processes are often too long to be used effectively in FISH, requiring partial digestion by restriction enzymes that are difficult to control); 3) targeted chromosome “painting” on a very fine level such that microduplications, microinversions and microdeletions can be detected (current techniques allow for painting of chromosomes in sections, however, the smallest unit that can be painted in one color is 10 megabases (10 mB)); and 4) utilization of standard laboratory equipment for the visual detection of signals such that special filters, software and processing steps are not required.

Thus, the instant method provides a method in which a single chromosomal region can be labeled with more than one color. For example, additional labels can be used to give more colors, e.g., 3 labels gives 7 distinguishable signals (the three individual colors, three combinations of two colors, and one combination of all three colors), four labels gives 15 distinguishable signals, and so on.

Detectable labels suitable for use in the present method, compositions and kits include any label detectable by spectroscopic, photochemical, biochemical, immunochemical, electrical, optical or chemical means. Useful labels include biotin for staining with labeled streptavidin conjugate, magnetic beads (e.g., DYNABEADS), fluorescent dyes (e.g., fluorescein, TEXAS RED, rhodamine, green fluorescent protein, cyanins and the like), radiolabels (e.g., 3H, ³⁵S, 14C, or ³²P, enzymes (e.g., horseradish peroxidase, alkaline phosphatase and others commonly used in ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241, which are herein incorporated by reference.

As noted above, an optically detectable signature refers to a light signal that can be detected by a fluorescence microscope, for example. An optically detectable signature may be made up of one or more signals, where the signal is produced by a label. An optically detectable signature includes: a single signal, a combination of two or more signals, ratio of magnitude of signals, etc. The signal may be visible light of a particular wavelength. An optically detectable signature may be provided by a fluorescent signal(s).

When more than one label is used, fluorescent moieties that emit different signal can be chosen such that each label can be distinctly visualized and quantitated. For example, a combination of the following fluorophores may be used: 7-amino-4-methylcoumarin-3-acetic acid (AMCA), TEXAS RED (Molecular Probes, Inc.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a, 4a diaza-3-indacenepropionic acid, eosin-5-isothiocyanate, erythrosin-5-isothiocyanate, and CASCADE BLUE acetylazide (Molecular Probes, Inc.). Hybridized oligonucleotides can be viewed with a fluorescence microscope and an appropriate filter for each fluorophore, or by using dual or triple band-pass filter sets to observe multiple fluorophores. See, e.g., U.S. Pat. No. 5,776,688.

While the methods are not so limited, methods for combinatorial labeling are described in, e.g., see, Ried et al., 1992, Proc. Natl. Acad. Sci. USA 89, 1388-1392; Tanke, H. J. et al., 1999, Eur. J. Hum. Genet. 7:2-11. By using combined binary ratio labeling (COBRA) in conjunction with highly discriminating optical filters and appropriate software, over 40 signals can be distinguished in the same sample, see, e.g., Wiegant, J. et al., 2000, Genome Research, 10(6):861-865 (48-color FISH is feasible and more FISH colors may be generated using fewer primary fluorophores); Szuhai, K. et al., 2006, Nat. Protoc. 1(1):264-75 (staining of all 24 human chromosomes is accomplished with only four fluorochromes); Karhu, R. et al., 2001, Genes Chromosomes Cancer, 30(1):105-9 (discussion of 42-color multicolor FISH technique permitting detection of chromosomal aberrations the resolution of chromosome arms); Rapp et al., 2006, Cytogenet Genome Res. 114:222-226 (review of practice and applications of COBRA-FISH).

Hybridized oligonucleotides also can be labeled with biotin, or digoxygenin, although secondary detection molecules or further processing may then be required to visualize the hybridized oligonucleotides and quantify the amount of hybridization. For example, an oligonucleotide labeled with biotin can be detected and quantitated using avidin conjugated to a detectable enzymatic marker such as alkaline phosphatase or horseradish peroxidase. Enzymatic markers can be detected and quantitated in standard calorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Prior to in situ hybridization, the oligonucleotides may be denatured. Denaturation is typically performed by incubating in the presence of high pH, heat (e.g., temperatures from about 70° C. to about 95° C.), organic solvents such as formamide and tetraalkylammonium halides, or combinations thereof.

Intact chromosomes are contacted with labeled amplification products under in situ hybridizing conditions. “In situ hybridizing conditions” are conditions that facilitate annealing between a nucleic aid and the complementary nucleic acid in the intact chromosomes. Hybridization conditions vary, depending on the concentrations, base compositions, complexities, and lengths of the probes, as well as salt concentrations, temperatures, and length of incubation. For example, in situ hybridizations may be performed in hybridization buffer containing 1-2×SSC, 50% formamide, and, in some but not all embodiment, blocking DNA to suppress non-specific hybridization. In general, hybridization conditions include temperatures of about 25° C. to about 55° C., and incubation times of about 0.5 hours to about 96 hours. Suitable hybridization conditions for a set of oligonucleotides and chromosomal target can be determined via experimentation which is routine for one of skill in the art.

The contacted sample can be read using a variety of different techniques, such as, for example, by microscopy, flow cytometry, fluorimetry, etc. Microscopy, such as, for example light microscopy, fluorescent microscopy or confocal microscopy, is an established analytical tool for detecting light signal(s) from a sample. In embodiments in which oligonucleotides are labeled with a fluorescent moiety, reading of the contacted sample to detect hybridization of labeled amplification products may be carried out by fluorescence microscopy. Fluorescent microscopy or confocal microscopy used in conjunction with fluorescent microscopy has an added advantage of distinguishing multiple labels even when the labels overlap spatially.

In certain embodiments, the label is a fluorescent dye. Fluorescent dyes (fluorophores) suitable for use as labels in the present method can be selected from any of the many dyes suitable for use in imaging applications, especially flow cytometry. A large number of dyes are commercially available from a variety of sources, such as, for example, Molecular Probes (Eugene, Oreg.) and Exciton (Dayton, Ohio), that provide great flexibility in selecting a set of dyes having the desired spectral properties. Examples of fluorophores include, but are not limited to, 4-acetamido-4′-isothiocyanatostilbene-2,2′ disulfonic acid; acridine and derivatives such as acridine, acridine orange, acridine yellow, acridine red, and acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-amino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy5, Cy5.5, and Cy7; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC(XRITC); fluorescamine; IR144; IR1446; Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (TEXAS RED), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; Alexa-Fluor dyes (e.g., Alexa Fluor 350, Alexa Fluor 430, Alexa Fluor 488, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, Alexa Fluor 750), Pacific Blue, Pacific Orange, Cascade Blue, Cascade Yellow; Quantum Dot dyes (Quantum Dot Corporation); Dylight dyes from Pierce (Rockford, Ill.), including Dylight 800, Dylight 680, Dylight 649, Dylight 633, Dylight 549, Dylight 488, Dylight 405; or combinations thereof. Other fluorophores or combinations thereof known to those skilled in the art may also be used, for example those available from Molecular Probes (Eugene, Oreg.) and Exciton (Dayton, Ohio). Quantum dots may also be employed.

Fluorescence of a hybridized chromosome can be evaluated using a fluorescent microscope. In general, excitation radiation, from an excitation source having a first wavelength, passes through excitation optics. The excitation optics causes the excitation radiation to excite the sample. In response, fluorescent molecules in the sample emit radiation that has a wavelength that is different from the excitation wavelength. Collection optics then collects the emission from the sample. The device can include a temperature controller to maintain the sample at a specific temperature while it is being scanned. A multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Table 1 below provides exemplary combinations of fluorophores that may be used together in combinations of 2, 3 or 4. This table is by no means comprehensive. In Table 1, different 2 dye combinations, 9 different 3 dye combinations, and 8 different 4 dye combinations are denoted (read vertically; filled-in black box indicates dyes in the combination).

TABLE 1
Exemplary Dye Combinations (AF = Alexa Fluor).

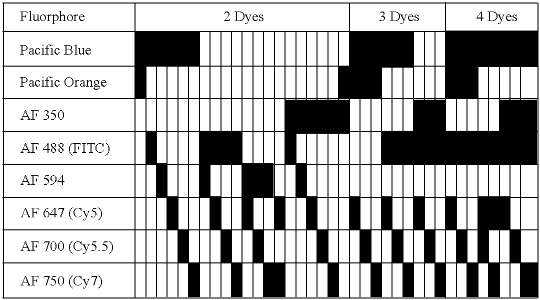

In general, cytogenetic data may be produced by any convenient method. In one embodiment, the staining method employed is a multicolor FISH-based method that allows the visualization of all 24 autosomes, each in a different color. Such “chromosome painting” approaches are reviewed in Speicher et al. (Nature Reviews (2005) 6: 782-792), Liehr et al. (Histol. Histopathol. (2004) 19:229-37) and Matthew et al. (Methods Mol. Biol. (2003) 220: 213-33) and include multiplex-FISH (M-FISH; Speicher et al., Nature Genet. (1996) 12: 368-375), spectral karyotyping (SKY; Schröck et al., Science (1996) 273: 494-497) and combined binary ratio labeling (COBRA; Tanke et al., Eur. J. Hum. Genet. (1999) 7: 2-11). Such methods provide for identification of intrachromosomal rearrangements, and may be performed on genomic samples from non-dividing or metaphase cells, for example. All such methods may be readily adapted for use herein.

In general, the in situ hybridization methods used herein include the steps of fixing an intact chromosome to a support, hybridizing the labeled amplification products to target nucleic acids in the intact chromosome, and washing to remove non-specific binding. In situ hybridization assays and methods for sample preparation are well known to those of skill in the art and need not be described in detail here.

In certain embodiments, the binding pattern of the labeled oligonucleotides to a chromosome may be compared with that of a reference chromosome. The reference chromosome may be from a supposedly healthy or wild-type organism. Briefly, the method comprises contacting under in situ hybridization conditions a test chromosome from the cellular sample with a plurality of fluorescently-labeled probes and contacting under in situ hybridization conditions a reference chromosome with the same plurality of fluorescently-labeled probes. After hybridization, the emission spectra created from the binding patterns from the test chromosome are compared to those of the reference chromosome.

Thus, the structure of a test chromosome may be determined by comparing the pattern of binding of the labeled oligonucleotides to the test chromosome with the binding pattern of the same labeled oligonucleotides with a reference chromosome. The binding pattern of the reference chromosome may be determined before, after or at the same time as the binding pattern for the test chromosome. This determination may be carried out either manually or in an automated system. In certain cases, the predetermined binding pattern may be determined experimentally or in silico. The binding pattern associated with the test chromosome can be compared to the binding pattern that would be expect for known deletions, insertions, translocation, fragile sites and other more complex rearrangements, and/or refined breakpoints. The matching may be performed by using computer-based analysis software known in the art. Determination of identity may be done manually (e.g., by viewing the data and comparing the signatures by hand), automatically (e.g., by employing data analysis software configured specifically to match optically detectable signature), or a combination thereof.

In another embodiment, the test sample is from an organism suspected to have cancer and the reference sample may comprise a negative control (non-cancerous) representing wild-type genomes and second test sample (or a positive control) representing a cancer associated with a known chromosomal rearrangement. In this embodiment, comparison of all these samples with each other using the subject method may reveal not only if the test sample yields a result that is different from the wild-type genome but also if the test sample may have the same or similar genomic rearrangements as another cancer test sample.

In certain embodiments, the subject method includes a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

A composition is also provided. In certain embodiments, the composition may comprise a plurality of sets of oligonucleotide probes, wherein: a) each set of oligonucleotide probes comprises at least 100 different oligonucleotide probes; b) the oligonucleotide probes of each set bind to a plurality of distinct non-contiguous regions of a reference chromosome; c) the plurality of sets of oligonucleotide probes bind to the reference chromosome in a predetermined binding pattern. Each set of labeled oligonucleotide probes may labeled so as to produce an optically detectable signature that is distinguishable from all other sets. The oligonucleotides may be in solution, or tethered to a solid support, e.g., in the form of an array, for example.

### Kits

Also provided by the subject invention is a kit for practicing the subject method, as described above. In certain cases, the subject kit contains a plurality of sets of oligonucleotide probes, wherein: a) each set of oligonucleotide probes comprises at least 100 different oligonucleotide probes; b) the oligonucleotide probes of each set bind to a plurality of distinct non-contiguous regions of a reference chromosome; c) the plurality of sets of oligonucleotide probes bind to the reference chromosome in a predetermined binding pattern. Each set of labeled oligonucleotide probes may be labeled so as to produce an optically detectable signature that is distinguishable from all other sets. The kit may further contain, materials for fluorescent labeling of oligonucleotides (e.g., fluorophores and enzymes, buffers, etc., for attaching the fluorophores to the oligonucleotides), reagents for in situ hybridization, and a reference sample to be employed in the subject method. The various components of the kit may be in separate vessels.

In addition to above-mentioned components, the subject kit may further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Utility

The subject method finds use in a variety of applications, where such applications generally include genomic DNA analysis applications in which the presence of a particular chromosomal rearrangement in a given sample is to be detected. The subject methods may also be used to finely map chromosomal breakpoints, and other aberrations, such as micro-inversions, deletions and translocations without a priori knowledge of their location.

In general, the methods involve the use of a set of labeled probes designed to anneal to a target chromosome, giving multi-color-coding at high density. The chromosome under study, which may or may not be suspected of containing a chromosomal rearrangement, is contacted with strand-specific labeled probes. After hybridization, the binding pattern of the probes is analyzed, as described above.

Specific analyte detection applications of interest include but are not limited to chromosomal rearrangements and aberrations. One embodiment of the genomic analysis assay allows the detection of a chromosome inversion. In this embodiment, the assay contacts probes specific for a region of a reference chromosomal region under in situ hybridization conditions. If the test chromosomal region contains an inverted chromosomal segment that is visualized by a specific alteration in the characteristic emission spectra, an inversion has occurred. Matching the location of a probe to a database may provide the nucleotide sequence information of the probe hybridized to the test chromosome. Using the sequence information, the detailed location of the inversion junction may be deciphered.

The subject methods also find utility in the detection of chromosomal rearrangements. In this embodiment, the assay contacts probes specific for a region of a reference chromosomal region under in situ hybridization conditions. If the test chromosomal region contains newly juxtaposed segments from distant chromosomal regions that are visualized by their characteristic emission spectra, a translocation or complex chromosomal aberration has occurred. Again, sequence information from a database describing the starting probes can be used to decipher the location of the translocation junction.

The subject methods find use in a variety of diagnostic and research purposes since chromosomal inversions and translocations play an important role in conditions relevant to human diseases and genomic evolution of many organisms.

In particular, the above-described methods may be employed to diagnose, or investigate various types of genetic abnormalities, cancer or other mammalian diseases, including but not limited to, leukemia; breast carcinoma; prostate cancer; Alzheimer's disease; Parkinson's disease; epilepsy; amyotrophic lateral sclerosis; multiple sclerosis; stroke; autism; Cri du chat (truncation on the short arm on chromosome 5), 1p36 deletion syndrome (loss of part of the short arm of chromosome 1), Angelman syndrome (loss of part of the long arm of chromosome 15); Prader-Willi syndrome (loss of part of the short arm of chromosome 15); acute lymphoblastic leukemia and more specifically, chronic myelogenous leukemia (translocation between chromosomes 9 and 22); Velocardiofacial syndrome (loss of part of the long arm of chromosome 22); Turner syndrome (single X chromosome); Klinefelter syndrome (an extra X chromosome); Edwards syndrome (trisomy of chromosome 18); Down syndrome (trisomy of chromosome 21); Patau syndrome (trisomy of chromosome 13); and trisomies 8, 9 and 16, which generally do not survive to birth.

The disease may be genetically inherited (germline mutation) or sporadic (somatic mutation). Many exemplary chromosomal rearrangements discussed herein are associated with and are thought to be a factor in producing these disorders. Knowing the type and the location of the chromosomal rearrangement may greatly aid the diagnosis, prognosis, and understanding of various mammalian diseases.

Certain of the above-described methods can also be used to detect diseased cells more easily than standard cytogenetic methods, which require dividing cells and require labor and time-intensive manual preparation and analysis of the slides by a technologist. The above-described methods do not require living cells and can be quantified automatically since a computer can be programmed to count the number and/or arrangement of fluorescent dots present.

The above-described methods can also be used to compare the genomes of two biological species in order to deduce evolutionary relationships.

Chromosomes may be isolated from a variety of sources, including tissue culture cells and mammalian subjects, e.g., human, primate, mouse or rat subjects. For example, chromosomes may be analyzed from less than five milliliters (mL) of peripheral blood. White blood cells contain chromosomes while red blood cells do not. Blood may be collected and combined with an anti-clotting agent such as sodium heparin. Chromosomes may also be analyzed from amniotic fluid, which contains fetal cells. Such cells can be grown in tissue culture so that dividing cells are available for chromosomal analysis within 5-10 days. Chromosomes may also be analyzed from bone marrow, which is useful for diagnosis of leukemia or other bone marrow cancers. Chromosomes may also be analyzed from solid tissue samples. A skin or other tissue biopsy in the range of about 2-3 mm may be obtained aseptically and transferred to a sterile vial containing sterile saline or tissue transport media to provide material for chromosome analysis. Fetal tissue obtained after a miscarriage can also be used for chromosome analysis, such as from the fetal side of the placenta, the periosteum overlying the sternum or fascia above the inguinal ligament, or from chorionic villi. Fetal tissue can also be collected from multiple sites such as the kidneys, thymus, lungs, diaphragm, muscles, tendons, and gonads. An amniocentesis may also be performed.

In addition to the above, the instant methods may also be performed on bone marrow smears, blood smears, paraffin embedded tissue preparations, enzymatically dissociated tissue samples, uncultured bone marrow, uncultured amniocytes and cytospin preparations, for example.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of sample analysis, comprising:
a) contacting a genomic sample comprising a test chromosome with a plurality of sets of labeled oligonucleotide probes under in situ hybridization conditions to produce a contacted sample having an oligonucleotide binding pattern, wherein:
i. each set of labeled oligonucleotide probes comprises at least 100 different oligonucleotide probes;
ii. the labeled oligonucleotide probes of each set bind to a plurality of distinct non-contiguous regions of a reference chromosome;
iii. said plurality of sets of labeled oligonucleotide probes bind to said reference chromosome in a predetermined binding pattern; and
iv. each set of labeled oligonucleotide probes is labeled so as to produce an optically detectable signature that is distinguishable from all other labeled sets;
b) imaging said contacted sample to provide an image showing said oligonucleotide binding pattern; and
c) analyzing said oligonucleotide binding pattern to identify a chromosomal rearrangement in said test chromosome relative to said reference chromosome.

2. The method of claim 1, wherein the analyzing step c) comprises:
comparing said oligonucleotide binding pattern with said predetermined binding pattern to identify said chromosomal rearrangement.

3. The method of claim 1, wherein said labeled oligonucleotide probes of each set are labeled with one or more fluorescent moieties.

4. The method of claim 1 wherein said optically detectable signature is produced by a single fluorescent moiety having a characteristic emission spectrum.

5. The method of claim 1 wherein said optically detectable signature is produced by two or more fluorescent moieties having characteristic emission spectrum.

6. The method of claim 2, wherein said imaging is carried out by using a fluorescence microscope.

7. The method of claim 3, wherein the distinct non-contiguous regions of said reference chromosome are bound by probes labeled with one or more fluorescent moieties having a single characteristic emission spectrum.

8. The method of claim 3, wherein the distinct non-contiguous regions of said reference chromosome are bound by probes labeled with one or more fluorescent moieties having multiple characteristic emission spectra.

9. The method of claim 3, wherein each chromosome region of said genomic sample is identifiable by its oligonucleotide binding pattern.

10. The method of claim 1, wherein the oligonucleotides of each set are selected to specifically hybridize to a region of said reference chromosome.

11. The method of claim 1, wherein the oligonucleotides of each set are tiled end to end.

12. The method of claim 1, wherein the oligonucleotides overlap with each other when bound to said test chromosome.

13. The method of claim 1, wherein the genomic sample comprises the entire complement of the chromosomal DNA from a mammalian cell.

14. The method of claim 13, wherein each chromosome region of said genomic sample is identifiable by its oligonucleotide binding pattern.

15. The method of claim 1, wherein said predetermined binding pattern of said reference chromosome is determined experimentally or in silico.

16. The method of claim 1, wherein said labeled oligonucleotide probes are between about 50 and 200 nucleotides in length.

17. A composition, comprising:
a plurality of sets of labeled oligonucleotide probes, wherein:
a) each set of labeled oligonucleotide probes comprises at least 100 different labeled oligonucleotide probes;
b) the labeled oligonucleotide probes of each set bind to a plurality of distinct non-contiguous regions of a reference chromosome;
c) said plurality of sets of labeled oligonucleotide probes bind to said reference chromosome in a predetermined binding pattern; and
d) each set of labeled oligonucleotide probes is labeled so as to produce an optically detectable signature that is distinguishable from all other sets.

18. The composition of claim 17, wherein said labeled oligonucleotide probes are tethered to a surface in the form of an array.

19. The composition of claim 17, wherein said labeled oligonucleotide probes are in solution.

20. A kit for analyzing a genomic sample according to claim 1, comprising:
a) a plurality of sets of labeled oligonucleotide probes; wherein
i. each set of labeled oligonucleotide probes comprises at least 100 different labeled oligonucleotide probes;
ii. the labeled oligonucleotide probes of each set bind to a plurality of distinct non-contiguous regions of a reference chromosome;
iii. said plurality of sets of labeled oligonucleotide probes bind to said reference chromosome in a predetermined binding pattern; and
iv. each set of labeled oligonucleotide probes is labeled so as to produce an optically detectable signature that is distinguishable from all other sets;
b) reagents for performing fluorescent in situ hybridization.
